(19) United States
(12) Patent Application Publication
(10) Pub. No.: US 2024/0157017 A1
(43) Pub. Date: May 16, 2024

(54) A MEDICAL DRESSING COMPRISING A BACTERIOSTATIC COMPOSITION

(71) Applicant: Mölnlycke Health Care AB, Göteborg (SE)

(72) Inventors: Marina Craig, Åsa (SE); Goran Edenro, Mölndal (SE); Eric Wellner, Göteborg (SE); Milja Elias, Skepplanda (SE); Sofia Almqvist, Göteborg (SE)

(21) Appl. No.: 18/552,455

(22) PCT Filed: Apr. 21, 2022
(86) PCT No.: PCT/EP2022/060492

§ 371 Date: Sep. 26, 2023

(30) Foreign Application Priority Data
Apr. 26, 2021 (EP) 21170460.6

(51) Int. Cl. A61L 15/44; A61K 31/44; A61L 15/28; A61L 15/58

(52) U.S. Cl. CPC A61L 15/44; A61K 31/44; A61L 15/28; A61L 15/58; A61L 2300/404

(57) ABSTRACT

Described is a medical dressing that includes a substrate. The dressing has a bacteriostatic composition integrated in the substrate or being provided as a coating on a surface of the substrate. The bacteriostatic composition can include deferiprone. Described is also a method for manufacturing a dressing and to the use of the medical dressing in preventing bacterial infections.

### TECHNICAL FIELD

The present disclosure generally relates to a medical dressing comprising a substrate. The medical dressing comprises a bacteriostatic composition integrated in the substrate or provided as a coating on a surface of the substrate. The present disclosure also relates to a method for manufacturing such a dressing and to the use of the medical dressing in preventing bacterial infections.

### BACKGROUND

Infection is a common problem in chronic and surgical wounds. A surgical site or an open wound is a suitable environment for bacteria to accommodate and colonize. A bacterial infection in the wound or at the skin surrounding the wound may disrupt the normal wound healing process and result in chronic, non-healing wounds.

Wound infecting bacteria often produce toxic substances, also known as virulence factors, which may damage the host tissue and allow the bacteria to establish at the wound site. Furthermore, wound infection is often associated with the formation of bacterial biofilms. Bacterial biofilms are clusters of bacteria that are attached to a surface and to each other and embedded in a self-produced matrix. The biofilm matrix comprises e.g. proteins, polysaccharides and extracellular DNA. Bacteria present in biofilms can employ various survival strategies to avoid the host immune system, such as staying inactive and “hidden” from the immune system, and at a later stage cause an acute infection. The bacteria may adapt to the biofilm environment, wherein e.g. the nutrient supply is more limited, and exhibit an altered gene expression and protein production. These adaptations can make the bacteria more resistant to antimicrobial therapy.

One common bacterial species in chronic wounds is Pseudomonas aeruginosa. P. aeruginosa produces several virulence factors, including i.a. pyoverdine and pyocyanine, and has the capacity to form biofilms on the skin. Such biofilms are typically difficult to manage and remove.

In wound treatment, antimicrobial agents are often used to eliminate or reduce the risk of infection of the wound. To that end, various types of antimicrobial dressings have been developed. Examples of antimicrobial agents that have been explored for use in wound dressings include conventional antiseptics, antibiotics, antimicrobial peptides, and metallic agents with antimicrobial properties. For example, dressings comprising silver-containing compounds, such as silver salts, incorporated in the dressing structure or provided as coatings on the dressing, are commonly used.

One concern with killing bacteria, which is the main purpose of an antimicrobial agent, is that the bacteria may develop a “defense” after long-term exposure, and thereby build up a tolerance against the used antimicrobial.

It would therefore be desirable to deceive and combat infectious bacteria in a different manner, particularly with an approach being suitable for long-term and repeated use.

Another challenge with antimicrobial dressings, and with dressings comprising skin- or wound beneficial agents in general, is to secure proper release of such agents from the dressing surface or dressing interior to ensure that the effect (e.g. the antimicrobial effect) actually takes place.

In view of this, there is a need to provide a dressing that alleviates the above mentioned challenges and that can be used to improve the infection prevention regimen in chronic as well as acute wounds.

### SUMMARY

In view of the above mentioned problems, it is an object of the present disclosure to provide improvements with respect to preventing infections in wounds, particularly chronic wounds.

According to a first aspect, there is provided a medical dressing comprising a substrate, wherein the dressing comprises a bacteriostatic composition; the bacteriostatic composition being integrated in the substrate and/or provided as a coating on at least a portion of a surface of the substrate, wherein the bacteriostatic composition comprises deferiprone.

The present disclosure is based on the realization that deferiprone can act as a bacteriostatic composition and control and inhibit the growth of bacteria commonly encountered in an infected wound. The inventors have found that the incorporation of deferiprone in a dressing can decrease toxic virulence factors produced by bacteria, e.g. pyoverdine (produced by P. aeruginosa), and also prevent the formation of biofilms.

By weakening or “disarming” the bacteria (instead of killing these), the virulent state of the bacteria may be changed into a less virulent phenotype. This way, the immune response can focus on healing and improving the wound status. Furthermore, without wishing to be bound by theory, it is believed that the approach of the present disclosure may prevent bacterial resistance, since the bacteria are managed in a manner that prevents the bacteria from becoming tolerated to the bacteriostatic composition, and furthermore secures that the immune response of a host can focus on battling the infection in a natural manner.

Deferiprone is a water soluble compound, known for its iron chelating effects. Iron chelators may reduce iron absorption by microorganisms, and thereby inhibit microbial growth and potentiate the antimicrobial activity of e.g. antibiotics. The inventors have evaluated various chelators, with the conclusion that deferiprone stands out as a compound being bacteriostatic per se. In other words, extracellular iron chelation is not the mechanism behind the surprising bacteriostatic effect observed with deferiprone.

The inventors have also found that deferiprone can be successfully introduced into a dressing, and also be released therefrom, which is normally a challenge when it comes to the incorporation of active agents into a dressing structure. The dressing of the present disclosure therefore provides a promising and commercially viable strategy for infection prevention in wound care, particularly in preventing the wound from turning into a chronic wound.

The bacteriostatic composition may either be incorporated into the substrate and/or provided as a coating on at least a portion of a surface of the substrate.

In embodiments, the substrate is a foam, preferably a polyurethane foam, an adhesive skin contact layer or wherein the substrate comprises absorbent gel forming fibers.

In embodiments, the bacteriostatic composition is provided as a coating on a skin-facing surface of the substrate.

Accordingly, the coating is arranged to be in direct contact with a wound and/or a surrounding dermal surface during use. The provision of the coating in direct contact with the wound may allow for a quicker bacteriostatic effect to be achieved.

As mentioned hereinbefore, the substrate may comprise absorbent gel forming fibers.

Absorbent gel forming fibers are fibers which, upon uptake of wound exudate become gelatinous and form a gel. The gel may retain and control exudate levels and thereby assist in maintaining a moist environment to promote wound healing and formation of granulation tissue. When the dressing of the present disclose comprises a substrate of absorbent gel forming fibers, typically no adhesive skin-contact layer is present. Such a dressing may be referred to as a “primary dressing” to be applied to the wound, but may require the application of a secondary dressing, such as an adhesive film dressing, to secure attachment to the dermal surface.

In embodiments, the absorbent gel forming fibers comprise polyvinyl alcohol (PVA), preferably crosslinked polyvinyl alcohol (PVA).

Such fibers are water soluble and capable of forming stable hydrogels upon contact with wound exudate. The integrity of the substrate is maintained even when large amounts of wound exudate have been absorbed.

When the substrate comprises gel forming fibers, the bacteriostatic composition is preferably provided as a coating on a surface of the substrate.

A substrate comprising gel forming fibers may be coated on both sides of the substrate; i.e. both on the skin-facing surface, as well as on a second surface, facing away from the skin or the wound. This way, a caregiver can apply either surface to the wound site and thereafter apply a secondary dressing, if desired.

If the substrate comprises absorbent gel forming fibers, a coating comprising the bacteriostatic composition and a non-aqueous solvent is generally preferred. Deferiprone is highly water soluble and the provision of a soluble aqueous coating onto a substrate comprising absorbent gel forming fibers may cause the fibers to start “gelling” during application of the coating, which is undesired.

Therefore, in embodiments, the bacteriostatic composition is provided as a coating, wherein the coating further comprises a non-aqueous solvent selected from methanol, ethanol, n-propanol, iso-propanol, n-butanol, s-butanol, and ethyl acetate. Preferably, ethanol is used as the non-aqueous solvent.

In embodiments, the bacteriostatic composition is provided as a coating, wherein the coating further comprises one or more cellulosic polymers selected from the group consisting of hydroxypropylmethylcellulose (HPMC), hydroxypropylcellulose (HPC), methylcellulose (MC), and ethylcellulose (EC).

The incorporation of cellulosic polymers in the coating is particularly beneficial when the coating is to be applied to a substrate comprising absorbent gel forming fibers. The cellulosic polymer can act as a thickener to provide a homogenous dispersion of the bacteriostatic agent, and thereby a homogenous coating.

It is, however, conceivable to incorporate one or more cellulosic polymer into a coating to be applied to a foam or an adhesive skin contact layer as well. The cellulosic polymer may, in such embodiments, be used to tailor the release of the bacteriostatic composition. In other words, the release of the bacteriostatic composition may be controlled by varying the amount of the cellulosic polymers.

In embodiments, the coating comprises:
0.5-14% by weight of deferiprone
1-15% by weight of a cellulosic polymers selected from the group consisting of hydroxypropylmethylcellulose (HPMC), hydroxypropylcellulose (HPC), methylcellulose (MC), and ethylcellulose (EC)
70-98% by weight of a non-aqueous solvent selected from methanol, ethanol, n-propanol, iso-propanol, n-butanol, s-butanol and ethyl acetate, and
0-15% by weight of water.

In such embodiments, deferiprone is typically provided as a dispersion in the coating.

In embodiments, the substrate is a foam.

Foam based dressings have the ability to absorb and handle large amounts of wound exudate, and may be utilized for wounds exuding medium to high amounts of exudate. A foam based dressing typically comprises an adhesive skin contact layer arranged to contact the skin of a wearer. If the dressing of the present disclosure comprises a foam substrate, the bacteriostatic composition may be coated on a surface of the foam, preferably on a surface that faces the skin of a wearer, e.g. on the surface facing the adhesive skin contact layer, where present. Alternatively, the bacteriostatic composition may be integrated in the foam by e.g. soaking the foam with the bacteriostatic composition or by adding the bacteriostatic composition during the foam polymerization reaction.

In embodiments, the substrate is a foam, and wherein the bacteriostatic composition is provided as a coating; the coating comprising a solvent selected from an alcohol, preferably methanol or ethanol, acetate, or an aqueous solution, such as water.

When such a coating has been applied to the surface of the foam, the coating is typically dried, prior to the optional assembly with additional layers or dressing components, e.g. an adhesive skin contact layer. During drying, the solvent used will evaporate quickly and efficiently, and the solvent used is selected such that it does not affect the properties of the foam. For example, it may prevent the foam from swelling during coating or during drying.

In alternative embodiments, the substrate is an adhesive skin contact layer.

A dressing comprising an adhesive skin contact layer, and, optionally a backing layer, is generally not designed to be absorbent, but may be useful in various applications. For example, people suffering from epidermolysis bullosa, a disease (or group of diseases) characterized by mechanical fragility of the skin and mucous membranes, may be treated by applying such dressings, preferably wherein the adhesive skin contact layer comprises a silicone based adhesive. The silicone based adhesive allows for non-traumatic removal of the dressing from the skin. Furthermore, infection prevention is an important element of the treatment of these patients.

When the substrate is an adhesive skin contact layer, the bacteriostatic composition may be provided as a coating on the skin-facing surface of the adhesive layer. Alternatively, or in addition, the bacteriostatic composition may be integrated into the adhesive skin contact layer. For example, the bacteriostatic composition may be homogenously dispersed in the adhesive skin contact layer. In such cases, the bacteriostatic composition may need a carrier or an excipient that enables a more homogenous distribution in the silicone based adhesive, and that also secures a controlled release of the composition.

In embodiments, the substrate is an adhesive skin contact layer, wherein the bacteriostatic composition is provided as a coating; the coating comprising an aqueous solvent, preferably water.

Accordingly, when the coating is exposed to wound exudate, it dissolves and the bacteriostatic composition is released to the wound site. A rapid bacteriostatic effect can thereby be achieved, and the bacteriostatic composition is released at steady concentrations. The release of the bacteriostatic composition will be substantially proportional to the amount of wound fluid exuded from the wound. The soluble coating is particularly advantageous for dressings, wherein the substrate is a foam or an adhesive skin contact layer.

In exemplary embodiments, the dressing comprises a backing layer, an adhesive skin contact layer and an absorbent pad arranged between the backing layer and the adhesive skin contact layer, and wherein the substrate is the adhesive skin contact layer; the bacteriostatic composition being provided as a coating on the skin-facing surface of the adhesive skin contact layer.

The pad may comprise a foam, e.g. a polyurethane foam as described hereinbefore.

Alternatively, or in addition, the pad may comprise a plurality of pad-forming layers, optionally wherein one of the pad-forming layers is a foam layer.

As mentioned hereinabove, the coating provided on the skin-facing surface of the adhesive skin contact layer may comprise an aqueous solvent, preferably water.

In exemplary embodiments, the bacteriostatic composition of the coating is a first bacteriostatic composition and wherein the absorbent pad and/or the adhesive skin contact layer comprises a second bacteriostatic composition.

The second bacteriostatic composition may comprise deferiprone. Alternatively, the second bacteriostatic composition comprises a different bacteriostatic composition.

The second bacteriostatic composition may be integrated in the absorbent pad or in a layer thereof. For example, the second bacteriostatic composition may be integrated in a foam layer comprised in the absorbent pad.

The concentration of the first, and the second bacteriostatic composition, respectively, may be the same or different.

By varying the concentration of the bacteriostatic composition within the dressing structure; i.e. within the absorbent pad and on the skin-facing surface, the release profile may be tailored to meet the specific requirements for various applications; e.g. certain types of wound and status of such wounds.

For example, it may, in certain embodiments, be beneficial to “boost” the bacteriostatic effect within the dressing, since a bacteriostatic composition inside the dressing structure typically has a longer distance to diffuse, and may require a certain amount of wound exudate to be absorbed compared to a bacteriostatic composition present in a coating provided on a skin-facing surface of the dressing. Accordingly, the absorbent pad may comprise more than one bacteriostatic composition or a higher concentration of the bacteriostatic composition.

According to another aspect of the present disclosure, there is provided a method for manufacturing a medical dressing comprising
a) providing a substrate, wherein the substrate is a foam, an adhesive skin contact layer or a substrate comprising absorbent gel-forming fibers,
b) providing a bacteriostatic composition in the form of a solution, suspension or a dispersion, wherein the bacteriostatic composition comprises deferiprone,
c) coating the bacteriostatic composition on at least a portion of a surface of the substrate, and/or
d) integrating the bacteriostatic composition in the substrate.

The method may further comprise the step of:
drying the coating on the surface of the substrate after step c).

During drying, the solvent typically evaporates. A release liner may be applied to the adhesive surface when the coating has been dried.

In embodiments, the bacteriostatic composition is coated on at least a portion of a surface of the substrate by means of spray coating.

Spray coating is a simple coating method associated with various advantages. For example, the fact that the bacteriostatic composition can be sprayed allows for a more controlled application of the coating. The area to be sprayed as well as the size of the droplets on the surface may be controlled. If the composition is spray coated on an adhesive skin contact layer, the coating preferably does not fully cover the adhesive layer, since this may adversely affect the adhesive properties of the adhesive layer.

The present disclosure also relates to a medical dressing as described hereinbefore for use in preventing bacterial infections.

According to another aspect, the present disclosure relates to the use of deferiprone as a bacteriostatic agent.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF DRAWINGS

The various aspects of the present disclosure, including its particular features and advantages, will be readily understood from the following detailed description and the accompanying drawings, in which:

FIG. 1a illustrates a schematic perspective view of a medical dressing according to an exemplary embodiment of the present disclosure comprising a substrate comprising absorbent gel forming fibers.

FIG. 1b illustrates a cross-sectional view along the line A-A in FIG. 1a.

FIG. 2a illustrates a schematic perspective view of a medical dressing according to an exemplary embodiment of the present disclosure comprising an absorbent foam.

FIG. 2b illustrates a cross-sectional view along the line A-A in FIG. 2a.

FIG. 2c illustrates an enlarged cross-sectional view of the cut-out Y in FIG. 2b.

FIG. 3a illustrates a cross-sectional view of a medical dressing according to an exemplary embodiment of the present disclosure comprising an adhesive skin contact layer.

FIG. 3b illustrates an enlarged cross-sectional view of the cut-out X in FIG. 3a.

FIG. 4 illustrates a schematic perspective view of a medical dressing according to an exemplary embodiment of the present disclosure.

FIG. 5 schematically outlines the steps of the method according to an exemplary embodiment of the present disclosure.

FIG. 6 illustrates the effect of deferiprone in simulated wound fluid (SWF) on the growth of P. aeruginosa (PaO1) in planktonic cultures with different bacterial start concentrations.

FIG. 7a illustrates the growth of P. aeruginosa (PaO1) at different time points in the presence of different concentrations of deferiprone in SWF.

FIG. 7b illustrates the attachment of P. aeruginosa (PaO1) to pegs at different time points in the presence of different concentrations of deferiprone in SWF.

FIG. 8 illustrates the effect of deferiprone on the growth of P. aeruginosa (PaO1) in a collagen gel with different bacterial start concentrations.

FIG. 9a illustrates clinical wound isolates of P. aeruginosa and virulence factor present (+) and absent (−).

FIG. 9b illustrates the growth of the clinical wound isolates of P. aeruginosa treated with deferiprone.

FIG. 10a illustrates the growth of Actinobacter baumanii treated with 3 mM deferiprone.

FIG. 10b illustrates the growth of Escherschia coli treated with 3 mM deferiprone.

FIG. 10c illustrates the growth of Klebsiella pneumoniae treated with 3 mM deferiprone.

FIG. 11a illustrates the growth of P. aeruginosa inside deferiprone impregnated foam prototypes.

FIG. 11b illustrates the growth of P. aeruginosa inside and outside deferiprone impregnated foam prototypes.

FIG. 12a illustrates the growth of P. aeruginosa inside and outside deferiprone impregnated foam prototypes containing different amounts of deferiprone in collagen matrix gels.

FIG. 12b illustrates the growth of P. aeruginosa inside and outside deferiprone impregnated PVA gelling fiber prototypes containing different amounts of deferiprone in collagen matrix gels.

FIG. 13a illustrates the growth of P. aeruginosa exposed to sub bacteriostatic concentrations of deferiprone in SWF after 72 hours.

FIG. 13b illustrates the effect on pyoverdine levels produced by P. aeruginosa exposed to sub bacteriostatic concentrations of deferiprone in SWF after 72 hours.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person.

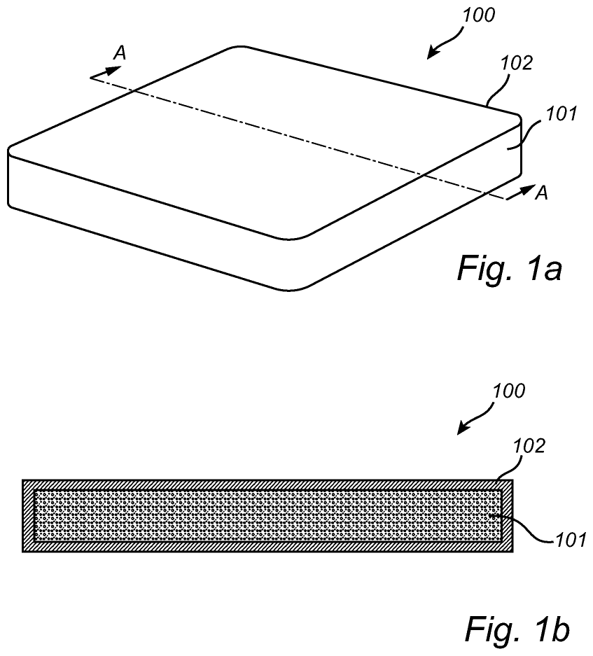

Various exemplary embodiments of medical dressings according to the present disclosure are conceptually illustrated in FIGS. 1a-b, 2a-c, 3a-b and 4.

The medical dressing comprises a substrate and a bacteriostatic composition being integrated in the substrate and/or provided as a coating on at least a portion of a surface of the substrate, wherein the bacteriostatic composition comprises deferiprone.

As used herein, the term “bacteriostatic composition” means a composition that prevents the growth of bacteria; i.e. a composition that keeps the bacteria in the stationary phase of growth. The bacteriostatic composition prevents the bacteria from reproducing, but does not kill them.

The term “bacteriostatic composition comprising deferiprone” means that the bacteriostatic composition may consist of deferiprone or that the bacteriostatic composition comprises deferiprone and, optionally, one or more additional bacteriostatic agent(s).

In embodiments, the concentration of deferiprone is from 0.1 to 20 mg/cm2, e.g. from 0.2 to 15 mg/cm2, e.g. from 0.3 to 5 mg/cm2.

The concentration may be different in cases where the bacteriostatic composition is provided as a coating on a surface of the substrate, and in cases where the bacteriostatic composition is integrated in the substrate.

In FIG. 1, the dressing 100 comprises a substrate comprising absorbent gel forming fibers. The substrate is denoted 101 in this figure.

As used herein, the term “gel forming fibers” means fibers that are water soluble and which, in contact with wound exudate, form a hydrogel.

The term “substrate comprising absorbent gel forming fibers” means that at least 75% of the substrate comprises gel forming fibers. In embodiments, the substrate consists of absorbent gel forming fibers.

The absorbent gel forming fibers preferably comprise polyvinyl alcohol (PVA), preferably crosslinked polyvinyl alcohol (PVA).

A substrate comprising gel forming fibers is typically combined with another, “secondary” dressing that may facilitate attachment to the skin or the wound of a patient.

When the substrate comprises gel forming fibers, the bacteriostatic composition is preferably provided as a coating 102 on at least one surface of the substrate, preferably as a coating on a skin-facing surface of the substrate 101. As illustrated in FIG. 1a and 1b, all surfaces of the substrate 101 are provided with the coating 102. The user can therefore choose either the top surface or the bottom surface for application onto a wound. The top or the bottom surface may thus form the skin-facing surface of the substrate.

A substrate comprising hydrophilic gel forming fibers is typically incompatible with aqueous solutions. Therefore, the bacteriostatic composition should be provided in a solvent system which does not impair or affect the gel forming fibers during application.

Accordingly, the coating 102 may comprise a non-aqueous solvent selected from methanol, ethanol, n-propanol, iso-propanol, n-butanol, s-butanol and ethyl acetate. Preferably, ethanol is used as the non-aqueous solvent.

The non-aqueous coating may be applied to the dressing 100 by impregnating the substrate 101 in a non-aqueous solution and subsequently drying the coating by conventional means. The entire outer surface of the substrate 101 may thus be provided with the bacteriostatic coating 102, as illustrated in FIG. 1b.

In embodiments, particularly where the substrate comprises absorbent gel forming fibers, the coating may comprise one or more cellulosic polymers selected from the group consisting of hydroxypropylmethylcellulose (HPMC), hydroxypropylcellulose (HPC), methylcellulose (MC), and ethylcellulose (EC).

The incorporation of a cellulosic polymer into a coating to be applied to a substrate comprising absorbent gel forming fibers facilitates the provision of a homogenous dispersion of the bacteriostatic composition. However, a cellulosic polymer may also be incorporated in a coating intended for other substrates, such as foams and adhesive skin contact layers. This way, the release of the bacteriostatic composition may be controlled and tailored for different application. A compound that has more or less solubility in an aqueous solution (e.g. wound exudate) may be used if the release should be prolonged.

For example, the coating 102 may comprise
0.5-14% by weight of deferiprone
1-15% by weight of a cellulosic polymers selected from the group consisting of hydroxypropylmethylcellulose (HPMC), hydroxypropylcellulose (HPC), methylcellulose (MC), and ethylcellulose (EC)
70-98% by weight of a non-aqueous solvent selected from methanol, ethanol, n-propanol, iso-propanol, n-butanol, s-butanol and ethyl acetate, and
0-15% by weight of water.

Since deferiprone is water soluble, a small amount of water may be needed to first dissolve deferiprone. However, the amount of water in the solvent should be less than 15% by weight, preferably less than 10% by weight, in order to prevent the fibers from gelling during application of the coating.

In embodiments, the coating 102 comprises 1-6% by weight of deferiprone.

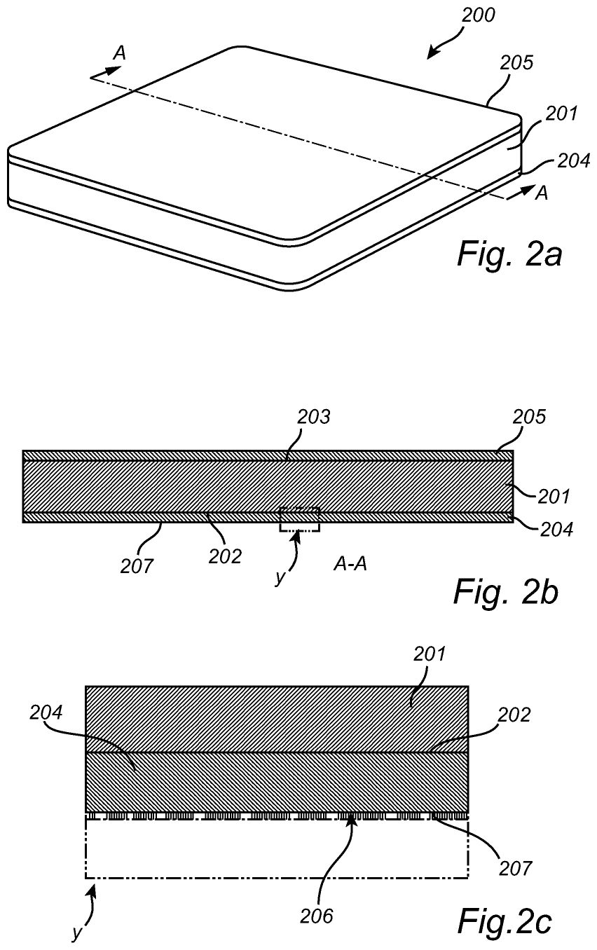

FIGS. 2a-2b illustrate a dressing 200 according to an exemplary embodiment of the present disclosure.

The dressing 200 comprises a backing layer 205, an adhesive skin contact layer 204 and an absorbent pad 201 arranged between the backing layer 205 and the adhesive skin contact layer 204.

The absorbent pad 201 may comprise a foam, e.g. a polyurethane foam. Accordingly, the substrate comprising the bacteriostatic composition may be the foam.

The foam is typically hydrophilic. Preferably, the foam is a hydrophilic polyurethane foam. An infected wound typically exudes large amounts of exudate, and the dressing must be capable of properly handling such exudate.

The polyurethane foam may e.g. be produced from a composition comprising a prepolymer based on hexamethylene diisocyanate (HDI), toluene diisocyanate (TDI) or methylene diphenyl diisocyanate (MDI).

The substrate comprising the bacteriostatic composition may be a foam comprised in the absorbent pad. In FIGS. 2a-2c, the absorbent pad is denoted 201. In FIGS. 2a-2c, the absorbent pad comprises only a foam; i.e. a foam layer. However, it is also conceivable that the absorbent pad comprises additional liquid handling layers.

In embodiments where the substrate comprising the bacteriostatic composition is a foam, the bacteriostatic composition may be distributed substantially homogenously within the foam. For example, the bacteriostatic composition may be in the form of a molecular dispersion or partial molecular dispersion within the foam 201. The term “molecular dispersion” means isolated molecules of the bacteriostatic composition. The term “partial molecular dispersion” means a plurality of isolated molecules as well as a plurality of isolated clusters of molecules, e.g. crystals or particles.

In embodiments, the bacteriostatic composition is chemically bound to the structure or internal surface, such as the pores, of the foam. The bacteriostatic composition may e.g. be bound to a charged internal surface of the foam.

It is also conceivable that the bacteriostatic composition is incorporated into the foam by adding the bacteriostatic composition to the pre-polymer before the foaming process step. This way, the bacteriostatic composition may become integrated into the foam and bound within the cell walls of the foam.

Alternatively, the foam is impregnated with the bacteriostatic composition. This way, a coating of the bacteriostatic composition onto a surface of the foam may be provided. Also, this mode of application may also provide a coating of the bacteriostatic composition on the internal pore surfaces of the foam.

As illustrated in FIG. 2b, the absorbent pad 201 has a first surface 202 and a second surface 203. The first surface 202 may be referred to as the bottom surface of the absorbent pad; i.e. the surface facing the wound or the skin of a wearer. The second surface 203 may be referred to as the top surface of the absorbent pad; i.e. the surface facing away from the wound or the skin of a wearer.

The adhesive skin contact layer 204 is attached to the first surface 202 of the absorbent pad 201; i.e. the foam substrate.

The medical dressing 200 may further comprise a backing layer 205. As illustrated in FIGS. 2a and 2b, the absorbent pad 201 is arranged between the adhesive skin contact layer 204 and the backing layer 205. Accordingly, the backing layer 205 is the outermost layer of the dressing. The backing layer 205 may be attached to the second surface 203 of the absorbent pad 201.

In the medical dressing illustrated in FIGS. 2a-2c, the substrate comprising the bacteriostatic composition may be the adhesive skin contact layer 204. The bacteriostatic composition is typically provided as a coating on a skin-facing surface of the adhesive skin contact layer 204.

The adhesive skin contact layer 204 preferably comprises a silicone based adhesive.

In FIG. 2c, a coating 206 comprising the bacteriostatic composition is illustrated on the skin-facing surface 207 of the adhesive skin contact layer 204. The coating 206 may be a discontinuous or continuous coating. In FIG. 2c, the coating is a discontinuous coating.

The coating 206 on the adhesive skin contact layer 204 typically comprises an aqueous solvent, and may e.g. be applied by means of spray coating.

In embodiments where the substrate is a foam, and wherein the bacteriostatic composition is provided as a coating, the coating typically comprises a solvent selected from an alcohol, preferably methanol or ethanol, acetate, or an aqueous solution, such as water.

The first (or the second) surface of the foam substrate may be coated prior to assembly of the foam substrate (i.e. the absorbent pad in FIG. 2) with the backing layer 205 and the adhesive skin contact layer 204. In such cases, the coating is applied to a surface of the foam, and subsequently dried such that the solvent used evaporates.

In embodiments, a first bacteriostatic composition is provided as a coating on the adhesive skin contact layer 204 and a second bacteriostatic composition is integrated in the absorbent pad; i.e. the foam 201.

Alternatively, or in addition, a second bacteriostatic composition is integrated in the adhesive skin contact layer 204. The first and the second bacteriostatic compositions may be the same or different. Depending on the type of wound or the mode of action of the dressing, the first and the second concentrations may be the same or different. For example, the second concentration of the bacteriostatic composition in the coating 206 may be lower than the first concentration of the bacteriostatic composition in absorbent pad; i.e. the foam 201.

The bacteriostatic composition in the foam will typically have a slower, more gradual, release profile than that of the coating, and may thus require higher concentrations of deferiprone to “boost” the release from the foam; i.e. from the interior of the dressing. There may, however, be situations and types of wounds where the opposite is beneficial.

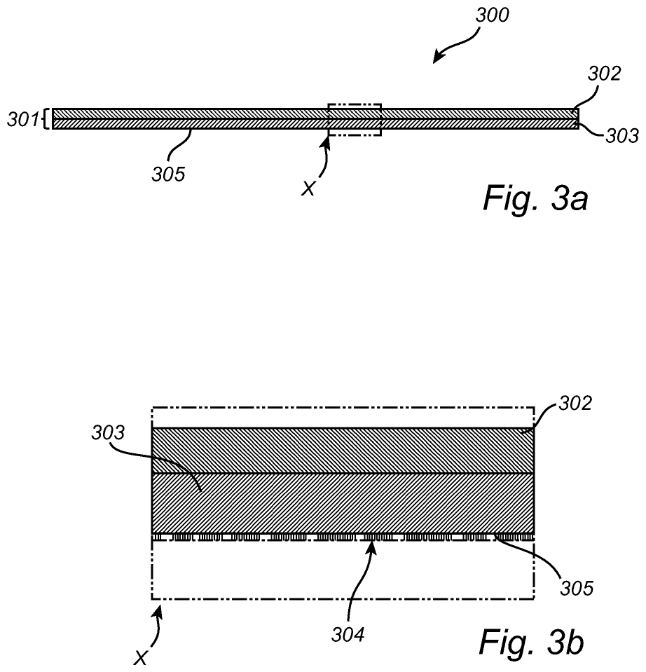

In FIGS. 3a and 3b, an alternative embodiment of the dressing of the present disclosure is conceptually illustrated. In this embodiment, the substrate comprising the bacteriostatic composition is an adhesive skin contact layer.

The adhesive skin contact layer illustrated in FIGS. 3a and 3b may form part of the dressings illustrated in FIGS. 2a-c and 4.

As used herein, the term “adhesive skin contact layer” means a layer configured to detachably adhere the dressing to a dermal surface. In other words, the adhesive skin contact layer is configured to contact the skin or the wound of a wearer. This layer may also be referred to as a “wound contact layer”. The adhesive skin contact layer may comprise one or more sub-layers. Preferably, the adhesive skin contact layer comprises a silicone based adhesive.

As illustrated in FIGS. 3a and 3b, the adhesive skin contact layer 301 comprises a polymeric film 302 and an adhesive silicone layer 303. The adhesive silicone layer 303 is arranged to contact the skin or the wound.

Accordingly, the adhesive skin contact layer 301 may be a laminate comprising at least one polymeric film 302 and an adhesive silicone layer 303.

The polymeric film 302 simplifies the manufacturing process, and provides stability and integrity to the adhesive skin contact layer 301. The polymeric film 302 is preferably a breathable film and may comprise e.g. polyethylene, polyamide, polyester or polyurethane. Typically, the polymeric film comprises polyurethane. The thickness of the polyurethane film may be from 15 to 100 μm, e.g. from 20 to 80 μm, preferably from 20 to 60 μm.

The bacteriostatic composition may either be integrated in the adhesive skin contact layer 301 or it may be provided as a coating 304 on the skin-facing surface 305 of the adhesive skin contact layer 301; i.e. on the silicone layer 303 (as best illustrated in FIG. 3b). It is also conceivable to apply a coating comprising the bacteriostatic composition on the polymeric film 302 prior to attachment with the silicone layer 303.

The dressing 300 may also comprise a backing layer (not shown) arranged on top of the adhesive skin contact layer.

When the bacteriostatic composition is provided as a coating, the coating 304 is preferably soluble in an aqueous medium. Accordingly, the coating dissolves in contact with wound exudate such that the bacteriostatic effect can be realized.

In embodiments where the bacteriostatic composition is integrated in the adhesive skin contact layer 301, the bacteriostatic composition may be a solid dispersion within the adhesive layer. The bacteriostatic composition may thus be distributed as a plurality of solid particles within the adhesive skin contact layer 301.

Alternatively, the bacteriostatic composition may be a molecular dispersion or a partial molecular dispersion within the adhesive skin contact layer 301.

In alternative embodiments, a first bacteriostatic composition is provided as a coating on the adhesive skin contact layer 301, and wherein a second bacteriostatic composition is integrated in the adhesive skin contact layer, wherein at least one of the first or the second bacteriostatic compositions comprises deferiprone.

As explained hereinbefore, the bacteriostatic composition may be provided as a coating on skin-facing surface of the substrate. Accordingly, the coating is configured to be in direct contact with the wound or a dermal surface.

The coating may be a discontinuous or a continuous coating on the skin-facing surface of the substrate. In other words, the coating may be a continuous layer on the surface of a plurality of sub-layered portions or dots of particles distributed on an area of the substrate surface.

In FIGS. 2c and 3b, the coating is a discontinuous coating, and the bacteriostatic composition is distributed in a discontinuous manner across the skin-facing surface of the dressing. In FIG. 1b, a continuous coating is illustrated.

In embodiments where the dressing comprises an adhesive skin contact layer, the coating may be discontinuous. This is to secure that the adhesive properties of the adhesive skin contact layer are not impaired.

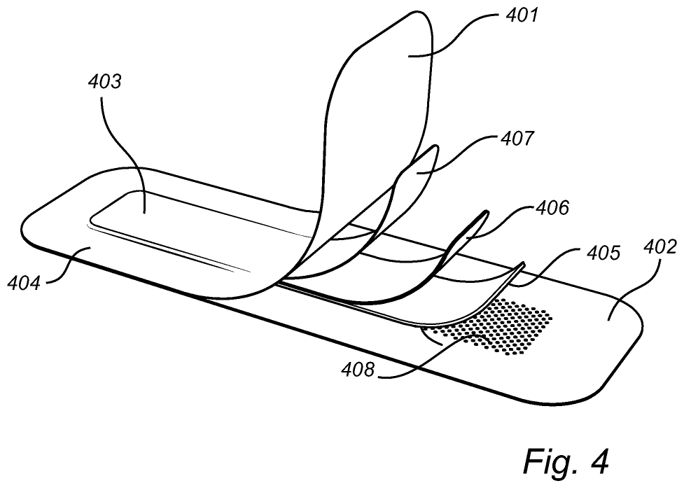

In FIG. 4, a so called “border dressing” is illustrated. The dressing comprises a backing layer 401, an adhesive skin contact layer 402 and an absorbent pad 403 arranged between the backing layer 401 and the adhesive skin contact layer 402. The backing layer 401 and the adhesive skin contact layer 402 are configured to extend beyond the contour of the absorbent pad 403 to form a border portion 404.

The absorbent pad 403 may be formed from a single layer or a plurality of pad-forming layers. For example, the absorbent pad may comprise a foam or a gel. It may also comprise a superabsorbent material e.g. superabsorbent polymers (SAP) or superabsorbent fibers (SAF).

In exemplary embodiments, the absorbent pad comprises two or more layers having different properties laminated together.

The absorbent pad 403 illustrated in FIG. 4 may comprise a first absorbent layer 405, a liquid distributing layer 406 and a second absorbent layer 407. Typically, the liquid distributing layer 406 is arranged between the first 405 and the second 407 absorbent layer, wherein the first absorbent layer 405 is the lowermost layer of the absorbent pad.

The first absorbent layer 405 may comprise a foam. Suitable foam materials for use in the first absorbent layer 405 include, but are not limited to polyurethane foams.

The second absorbent layer 407 may be a superabsorbent layer.

The superabsorbent layer may comprise a superabsorbent polymer (SAP) or superabsorbent fibers. A “superabsorbent polymer” or “SAP” is a polymer that can absorb up to 300 times its own weight in aqueous fluids. Superabsorbent polymers are constituted by water-swellable and water insoluble polymers capable of absorbing large quantities of fluid upon formation of a hydrogel. The SAP material may be in the form of particles, fibers, flakes or similar.

The liquid distributing layer 406 may comprise any material having the ability to distribute the exudate in an efficient manner. For example, the liquid distributing layer 406 may comprise a nonwoven material. A nonwoven imparts an appropriately balanced rigidity to the layer and to the dressing as such. It may also efficiently distribute and spread liquid absorbed by the absorbent layer 405 such that it can be evaporated through the backing layer 401 over a large surface. For example, the nonwoven may comprise viscose, polyester or blends thereof.

The layers can be joined by adhesion, lamination, using e.g. pressure and heat.

The absorbent pad may comprise additional layers, such as liquid transport layers, various combinations of foam and nonwoven layers laminated together.

With reference to FIG. 4, the layer 405 may comprises an absorbent foam, the layer 406 may be a liquid acquisition layer, and the layer 407 may be a superabsorbent layer.

Such a layered pad construction prevents accumulation of body liquids close to the skin, and improves the liquid handling of the dressing. Most wounds will contain some exudate, but the level of exudate may vary. In a chronic wound, the exudate production may be very large due to an ongoing inflammation. A dressing having the construction as explained above is suitable for handling large amounts of exudate, and prevents maceration of the skin surrounding the wound.

The bacteriostatic composition may be integrated in a layer of the absorbent pad 403. In embodiments, the bacteriostatic composition is provided as a coating on a layer of the absorbent pad, optionally prior to joining or lamination with one or more pad-forming layers or other layers of the pad 403.

In embodiments, at least two of the layers of the absorbent pad 403 comprise a bacteriostatic composition.

Alternatively, or in addition, the skin-facing surface of the adhesive skin contact layer 402 is coated with the bacteriostatic composition.

The bacteriostatic composition may be referred to as a first bacteriostatic composition, and the dressing may further comprise at least a second bacteriostatic composition.

As illustrated in FIG. 4, the adhesive skin contact layer 402 comprises a plurality of apertures 408. The apertures 408 extend through the adhesive skin contact layer 402. The apertures 408 allow for a quick absorption into the pad 403 without compromising the tight fit to the skin provided by the adhesive layer 402. The adhesive skin contact layer 402 comprises a plurality of apertures 408 in the area underlying the absorbent pad 403, but is void of apertures in the area forming the border portion 404. The lack of apertures in the border portion of the dressing is beneficial to improve the adhesion at the border portion 404 of the dressing and thereby improve the stay-on ability of the dressing.

It is beneficial to have an even distribution of adhesive over the surface of the pad 403 in order to keep the dressing in place during use.

The apertures 408 may have different shapes and densities along varying regions of the adhesive skin contact layer 402, and may be arranged in a regular or irregular pattern.

A coating comprising a bacteriostatic composition is typically provided on the non-apertured parts of the adhesive skin contact layer 402.

In the various embodiments described hereinbefore, the backing layer may be a thin film, sheet or membrane that is vapor permeable. Examples of suitable materials for the backing layer include, but are not limited to polyurethane, polyethylene or polyamide films, silicone films, polyester based nonwoven materials, and laminates of polyester-based nonwoven materials and polyurethane films. Suitably, the backing layer is a polyurethane film having a thickness of from 5 to 40 μm, e.g. from 15 to 25 μm.

In the various embodiments described, the term “skin contact layer” means a layer that is in contact with the skin of a wearer. The skin contact layer is adapted to adhere to the skin, which may or may not comprise a wound. The adhesive skin contact layer preferably comprises a silicone based adhesive. Such an adhesive is skin-friendly and permits the removal of the dressing without causing damage to the skin.

Examples of suitable silicone gels for use in the silicone based adhesive of the of the adhesive skin contact layer (204, 301) as described with reference to FIGS. 2, 3 and 4 include the two component RTV systems, such as Liveo MG-7-9960 (DuPont), and SilGel 612 (Wacker Chemie AG) mentioned herein, as well as NuSil silicone elastomers. In embodiments of the invention the adhesive may comprise a soft silicone gel having a softness (penetration) of from 8 to 22 mm, e.g. from 12 to 17 mm, as measured by a method based on ASTM D 937 and DIN 51580, the method being described in European Patent Application No 14194054.4. The thickness of the adhesive skin contact layer is typically at least 20 μm. The thickness of the adhesive skin contact layer may be from 30 to 200 μm.

The silicone based adhesive may be coated onto the polyurethane foam in FIG. 2. Alternatively, it may be attached to a polymeric film as described with reference to the dressing in FIG. 3 and FIG. 4.

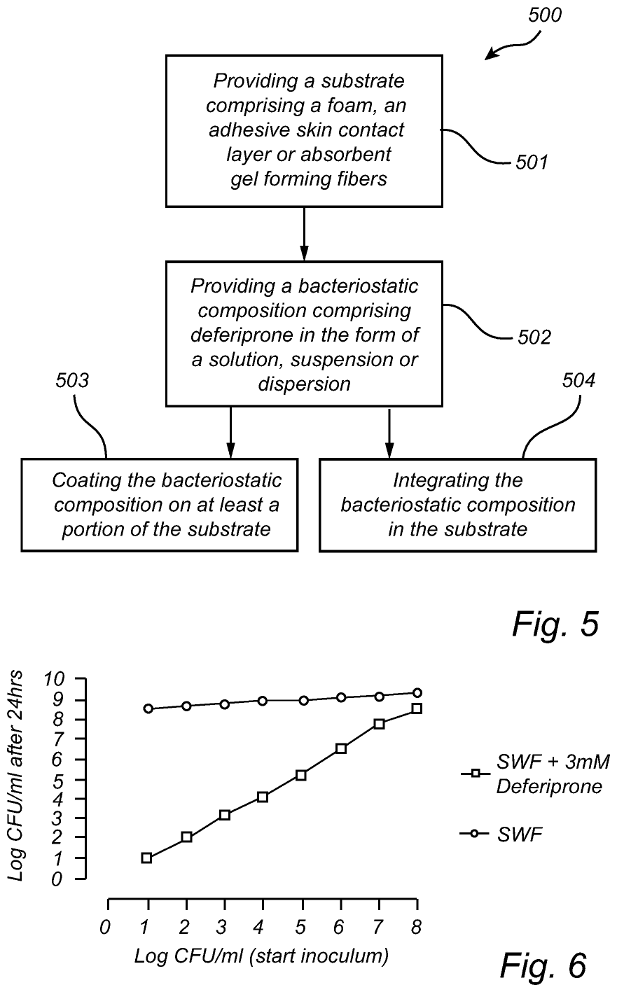

With reference to FIG. 5, a second aspect of the present disclosure is schematically illustrated, covering a method for manufacturing a medical dressing.

The method comprises:
providing a substrate, wherein the substrate is a foam, an adhesive skin contact layer or a substrate comprising absorbent gel-forming fibers (step 501),
providing a bacteriostatic composition in the form of a solution, suspension or a dispersion, wherein the bacteriostatic composition comprises deferiprone (step 502),
coating the bacteriostatic composition on at least a portion of a surface of the substrate (step 503), and/or
integrating the bacteriostatic composition in the substrate (step 504).

Depending on the substrate used, the coating may be soluble, partially soluble or non-soluble in an aqueous medium.

In embodiments where the substrate comprises an absorbent foam, the bacteriostatic composition may be provided by dissolving deferiprone in a solvent selected from an alcohol, preferably methanol or ethanol, acetate, or an aqueous solvent, preferably water. The bacteriostatic composition is subsequently coated on a surface of the foam.

In situations where the bacteriostatic composition is integrated in the foam substrate (step 504), when the substrate comprises or consists of a hydrophilic foam, e.g. a polyurethane foam, the bacteriostatic composition may be added to or mixed with a prepolymer before the foaming process step. This way, the bacteriostatic composition may become integrated into the foam and bound within the cell walls of the foam.

In embodiments where the substrate comprises a silicone based adhesive skin-contact layer, the bacteriostatic composition may be provided by dissolving deferiprone in an aqueous medium, preferably water. The bacteriostatic composition is subsequently coated on the skin-facing surface of the silicone based adhesive skin contact layer.

In embodiments where the bacteriostatic composition is integrated in the silicone based skin contact layer (step 504), the bacteriostatic composition may be added to an uncured mixture of silicone gel adhesive, and the adhesive mixture is subsequently cured.

In embodiments where the substrate comprises absorbent gel-forming fibers, the bacteriostatic composition is provided in a non-aqueous solvent system and subsequently coated on the substrate. Typically, the coating is a continuous coating covering all surfaces of the substrate.

For example, the bacteriostatic composition may be provided as a dispersion by mixing 0.5-14% by weight, e.g. 1-6% by weight of deferiprone with:
1-15% by weight of a cellulosic polymers selected from the group consisting of hydroxypropylmethylcellulose (HPMC), hydroxypropylcellulose (HPC), methylcellulose (MC), and ethylcellulose (EC)
70-98% by weight of a non-aqueous solvent selected from methanol, ethanol, n-propanol, iso-propanol, n-butanol, s-butanol and ethyl acetate, and
0-15% by weight of water.

A relatively high amount of the non-aqueous solvent is required to prevent the absorbent gel forming fibers from gelling during application of the coating. A small amount of water may be required since deferiprone is a water soluble agent. The coating may be applied by soaking or dipping the substrate in the coating solvent system.

The step of coating (503) is not limited to a specific coating method, but any coating means may be utilized.

After the coating step, the method further comprises the step of
drying the coating on the surface of the substrate.

Drying is performed by means well known to the skilled person.

In embodiments, the bacteriostatic composition is coated on at least a portion of the substrate by means of spray coating.

This coating technique is beneficial as it allows for flexibility depending on the dressing or substrate to be used and depending on the type of wound to be treated. It is also a simple means to apply the coating.

Spray coating is preferably used to coat the adhesive skin contact layer; i.e. the layer to be arranged in contact with the wound or the skin. In such cases, selected areas of the adhesive layer may be coated and the size of the droplets on the surface may be controlled to avoid interfering with the adhesive properties of the adhesive layer.

In another aspect, the present disclosure covers the use of deferiprone as a bacteriostatic agent.

### EXAMPLES

### Example 1: Bacteriostatic Effect of Deferiprone on Planktonic Cultures of Pseudomonas Aeruginosa, PaO1 with Different Bacterial Inoculum Sizes

An evaluation of possible bacteriostatic effect of deferiprone (3 mM) on the gram-negative bacterium Pseudomonas aeruginosa (ATCC #15692, PaO1) and effect of bacterial start concentrations was performed. Deferiprone (3 mM) was dissolved into Simulated Wound Fluid (SWF) (fetal bovine serum (FBS) and Peptone Water (PW) mixed in equal proportions, which correspond to the protein and electrolyte concentration of wound exudates (Emiko Aiba-Kojima, MD et.al Wound Rep Reg (2007) 15 511-520; Trengove, N et al Wound Rep Reg (1996) 4 1067-1927)). Bacterial concentrations ranging from 10−1×10⁸ CFU/ml were inoculated into different test tubes +/− Deferiprone (3 mM) in SWF and incubated at 35° C. for 24 hrs before bacterial numbers were determined using petrifilm.

As illustrated in FIG. 6, a true bacteriostatic effect was observed at 3 mM of deferiprone. The observed effect is not changed when using different start inoculums, showing that 3 mM deferiprone is non-toxic to PaO1. Bacteria grown in SWF without deferiprone grows to ˜1×10⁹ CFU/ml regardless of bacterial start concentrations. In the presence of 3 mM deferiprone the bacterial concentration after 24 hrs equals the bacterial start concentration. The mechanism observed is different from bacteriostatic antibiotics, where changes in inoculum often changes the Minimal Inhibitory Concentration (MIC). This experiment shows that an increase in bacterial start concentrations does not alter the bacteriostatic effects observed.

### Example 2: Bacteriostatic Effect of Deferiprone on Pseudomonas Aeruginosa and Biofilm Formation on Plastic Surfaces

An evaluation of possible bacteriostatic effect of deferiprone (3-6 mM) on the gram-negative bacterium Pseudomonas aeruginosa (ATCC #15692, PaO1) and connection to ability to form biofilm on a plastic surface, using the Calgary biofilm device (CBD) (Journal of Clinical Microbiology, June 1999, p. 1771-1776) was performed. This method utilizes 96-well microplates with lids containing 96 plastic cone shaped protrusions that extend into the wells of the plate. By submerging the plastic cones into bacterial solution, biofilm attachment to the pegs can be studied. Different concentrations of Deferiprone (0, 0.37, 0.75, 1.5, 3, 6 mM) were dissolved in Simulated Wound Fluid (SWF) and added to wells in the CBD plate. PaO1 was then inoculated at a total bacterial concentration of 1×10⁶ CFU/ml and the lid was placed on the plate. Following incubation at 35° C. for 24, 48, 72 hrs, bacterial concentration from the wells were calculated using petrifilm and the plastic protrusions were analyzed for biofilm attachment using a luminescence kit that quantifies bacterial viability (Promega BacTiter-GLO).

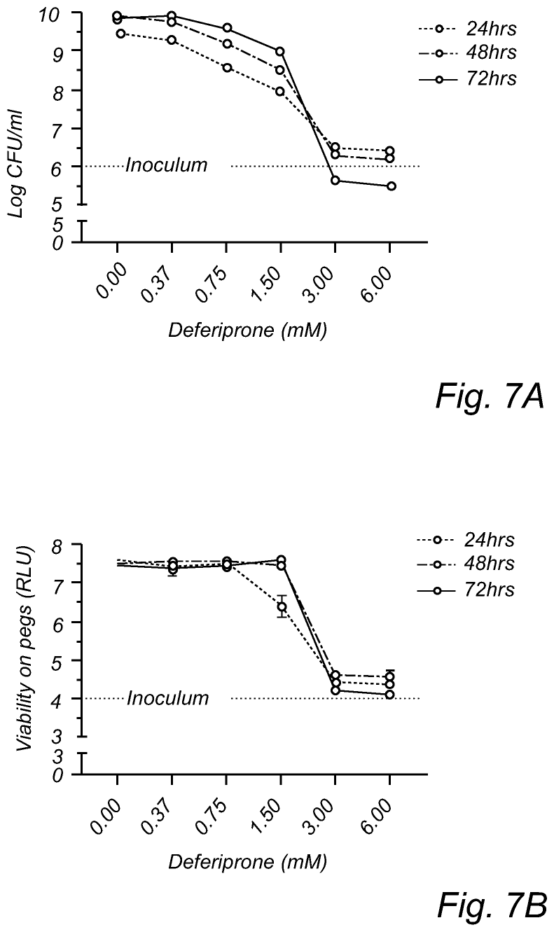

As illustrated in FIGS. 7a, 3 and 6 mM deferiprone is bacteriostatic, as the bacterial concentrations correspond to start inoculum (1×10⁶ CFU/ml) at all timepoints (24, 48, 72 hrs), which shows that the effect is truly bacteriostatic. This is unexpected for Pseudomonas aeruginosa since typically, this species has to be killed during treatment, otherwise full growth is obtained within 24 hrs. As illustrated in FIG. 7a, the bacterial concentrations are around inoculum concentrations for up to at least 72 hrs.

No biofilm was formed on the pegs of the CBD plate after 24, 48, 72 hrs at concentrations of 3 and 6 mM of deferiprone, as can be seen in FIG. 7b. The Promega kit used to look at bacterial viability is not as sensitive as using petrifilm, but it can distinguish between growth and no growth. The read-out signal for the pegs at 3, 6 mM equals the signal of the background which shows that there are no bacteria present on the pegs. At all other concentrations, a signal showing full growth is obtained (signals equal control pegs with full growth).

### Example 3: Bacteriostatic Effect of Deferiprone in Collagen (Wound-Like Matrix) Biofilms

In order to evaluate the bacteriostatic effects of deferiprone (3 mM) in a more “wound-like” matrix, experiments were performed in collagen-based matrix using PaO1 in a biofilm phenotype as opposed to planktonically grown bacterium. Collagen gels were created and inoculated with 1×10⁶ CFU/ml PaO1. Gels were incubated for 24 hrs at 35° C. in order to obtain bacteria in biofilm phenotype. New collagen gels +/−3 mM deferiprone were created and different bacterial start concentrations were inoculated into the gels. Following 24 hrs incubation at 35° C., the gels were solubilized and bacterial counts were measured using petrifilm.

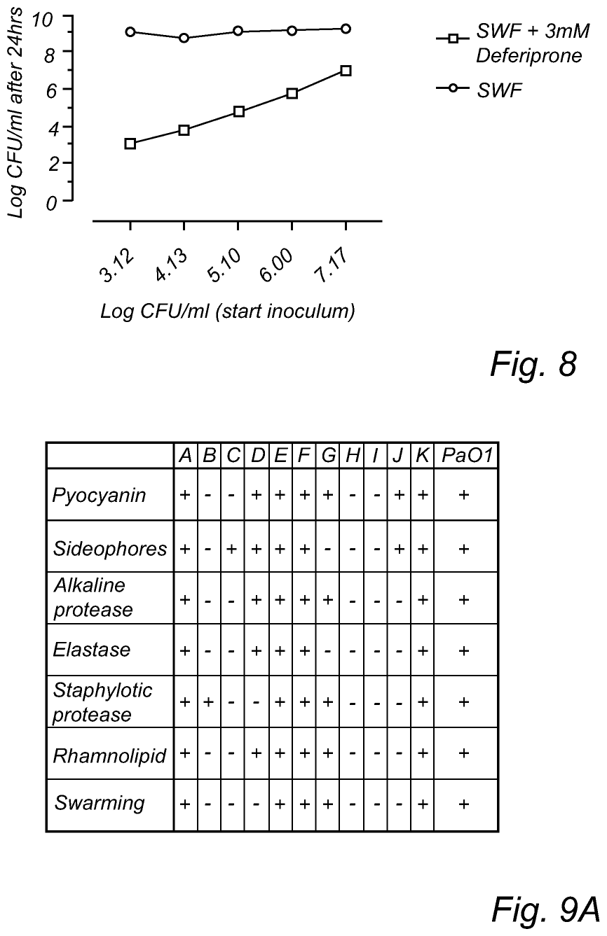

The results illustrated in FIG. 8 show that the same bacteriostatic effects seen with planktonic bacteria are observed when using bacteria in a biofilm phenotype. In the absence of deferiprone, normal growth is observed regardless of start inoculum. When deferiprone is present, a bacteriostatic effect is observed. Accordingly, biofilm derived bacteria are also bacteriostatic and thus the bacteriostatic effect is not dependent of state of phenotype for PaO1. This experiment confirms that deferiprone is able to exert bacteriostatic effects on PaO1 in a complex matrix, corresponding to a more wound-like situation.

### Example 4: Effect of Deferiprone on Different Clinical Wound Isolates of Pseudomonas Aeruginosa

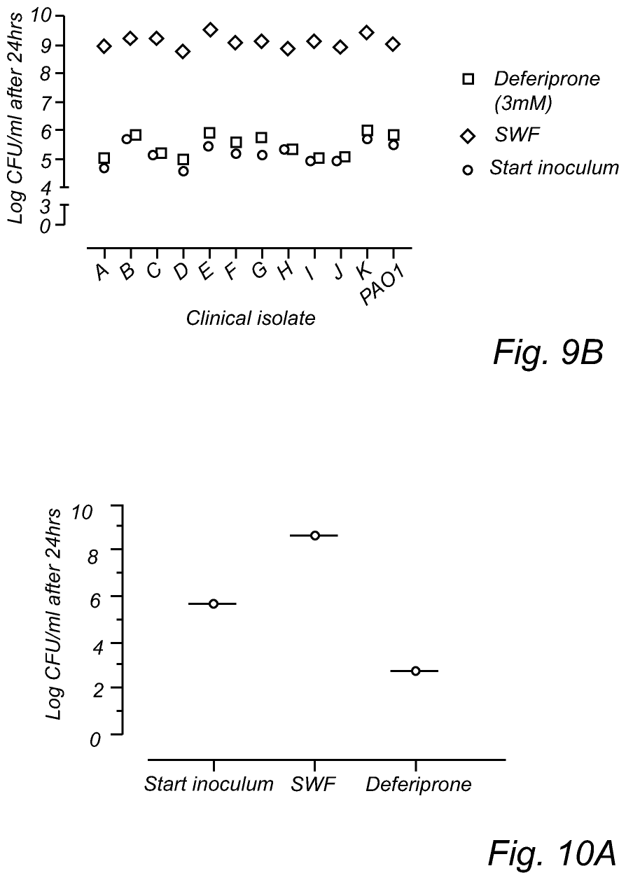

Bacteriostatic effect of deferiprone was tested on different clinical wound isolates of Pseudomonas aeruginosa. Isolates were typed according to known virulence factors (FIG. 9a) and subjected for treatment +/−3 mM deferiprone in SWF for 24 hrs at 35° C. Bacterial start concentrations and concentrations after treatment were measured using petrifilm.

In the absence of deferiprone, all clinical isolates showed characteristic growth, reaching ˜1×10⁹ CFU/ml (FIG. 9b). All isolates tested show bacteriostatic effects of deferiprone at 3 mM, regardless of virulence factor expression patterns. This shows that the virulence factor composition of the different strains is not related to the bacteriostatic effects observed with 3 mM deferiprone and that the effect observed in the laboratory Pseudomonas aeruginosa strain PaO1 extends to encompass clinical wound isolates.

### Example 5: Bacteriostatic Effect of Deferiprone on Other Gram-Negative Species

The bacteriostatic effect of deferiprone was tested on other gram negative species than Pseudomonas aeruginosa in order to see if the bacteriostatic effect of deferiprone extends to other gram negative species. The following bacteria were tested: Actinobacter baumannii, Echerschia coli, Klebsiella pneumoniae +/−3 mM deferiprone in SWF for 24 hrs at 35° C. Bacterial start concentrations and concentrations after treatment were measured using petrifilm.

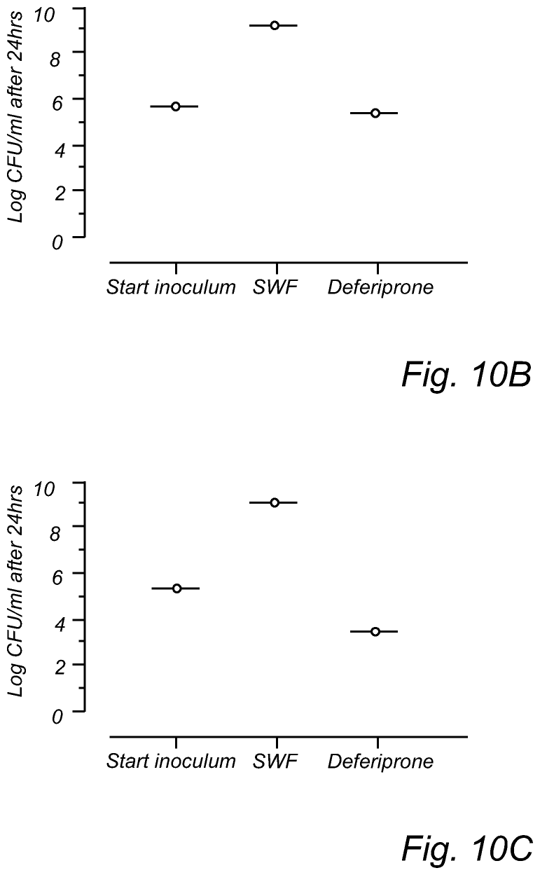

Echerschia coli showed the same bacteriostatic effects as previously shown with PaO1 (FIG. 10b). A. baumannii and K. pneumoniae are also sensitive to deferiprone (FIGS. 10a, and c, respectively).

### Example 6: Deferiprone-Impregnated Foam Prototypes and Effect on PaO1

Polyurethane foam prototypes were tested for bacteriostatic effects after impregnation with deferiprone.

Impregnated or imbibed foam was cut into circular pieces and then added either 0.5 or 1 ml of 1×10⁵ CFU/ml PaO1 in SWF, incubated for 24 hrs at 35° C. before bacterial counts were determined using petrifilms. In order to examine how the foam structure impregnated with deferiprone behaved, submaximal absorption volumes of bacterial solution was used (0.5 ml). After 24 hrs, the foam was firmly pressed in order to examine bacterial counts of the solution residing inside the prototype. In order to see if the constructed foam prototypes can release deferiprone, supramaximal absorption volume was used (1.0 ml) and bacterial counts were determined in the outside solution after 24 hrs.

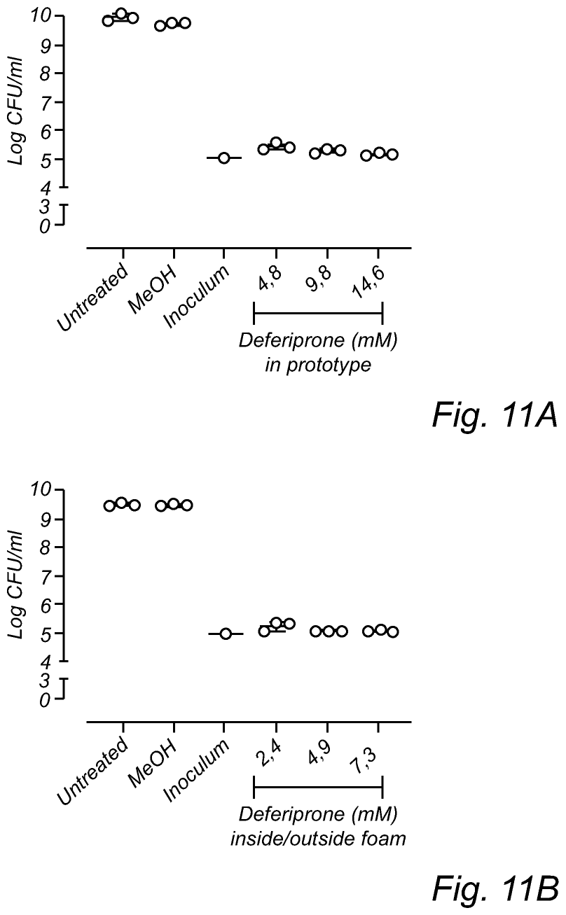

As illustrated in FIG. 11a, bacteria residing inside the foam prototypes are bacteriostatic, showing that the physical structure of the foam does not affect the ability of deferiprone to exert bacteriostatic effects on PaO1 inside the prototype. FIG. 11b extends these findings, showing that deferiprone is released from the prototypes into the surrounding bacterial solution and has a bacteriostatic effect. Since methanol was used in the impregnation process of the prototypes, possible traces of methanol and effects on bacterial growth on the prototypes was assessed with a methanol control, showing that methanol did not affect bacterial growth.

### Example 7: Effects of Deferiprone-Impregnated Foam and PVA-Gelling Fiber Prototypes on PaO1 Growth in a Wound-Like Collagen Matrix

The foam-impregnated prototypes used in example 6 were tested for effects on a more wound-like matrix consisting of collagen. In addition, PVA-gelling fiber prototypes impregnated with deferiprone were also tested in the same set up. Collagen gels were created and inoculated with 1×10⁵ CFU/ml PaO1 and prototypes were added on top of the gels before incubation for 24 hrs at 35° C. After removal of the prototypes, the collagen gels were solubilized and bacterial counts were determined. Methanol was included as control as previously explained.

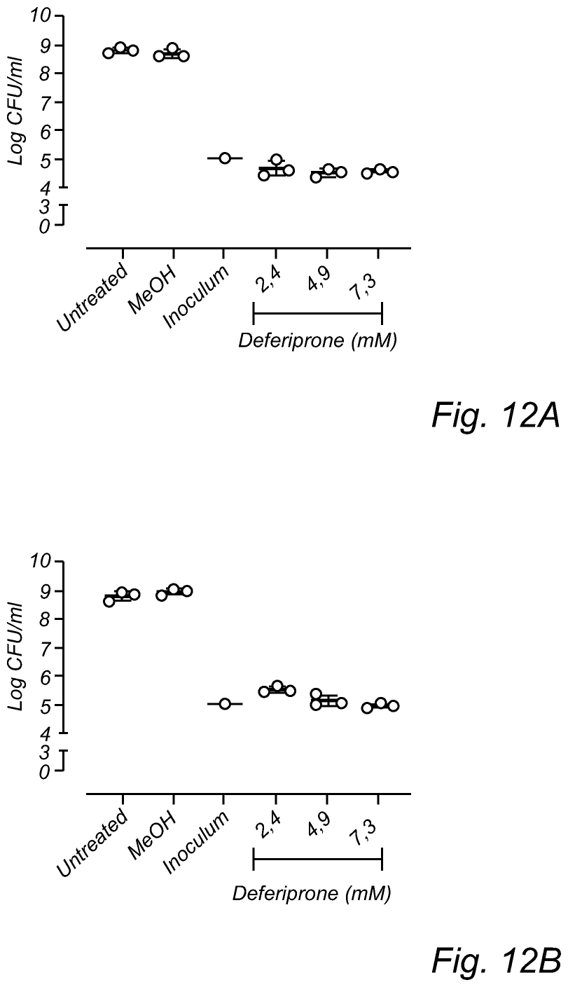

As shown in FIG. 12a-b, both the foam-impregnated prototypes, as well as PVA-gelling fiber prototypes are able to release deferiprone as bacteriostatic effects are observed for all tested prototypes.

### Example 8: Effect on the Virulence Factor Pyoverdine by Sub Bacteriostatic Concentrations of Deferiprone

In order to evaluate if sub-bacteriostatic concentrations (<3 mM) of deferiprone have an effect on the virulence factor pyoverdine, supernatants of planktonically grown PaO1 (1×10⁶ CFU/ml) +/− deferiprone at 0.3 and 0.03 mM in SWF were analyzed by LC-MS for pyoverdine after 72 hrs at 35° C.

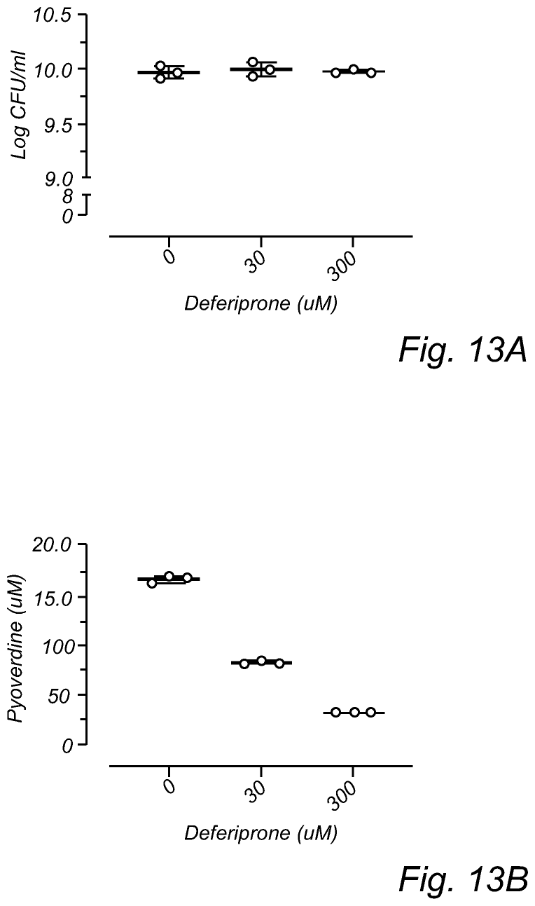

FIGS. 13a-b shows the results of bacterial growth and pyoverdine levels measured. 0.03 and 0.3 mM deferiprone does not affect growth characteristics of PaO1 (FIG. 12a). However, a dose-dependent decrease in pyoverdine levels were detected by LC-MS in the supernatants (FIG. 12b).

Terms, definitions and embodiments of all aspects of the present disclosure apply mutatis mutandis to the other aspects of the present disclosure.

Even though the present disclosure has been described with reference to specific exemplifying embodiments thereof, many different alterations, modifications and the like will become apparent for those skilled in the art.

Variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A medical dressing comprising a substrate, wherein said dressing comprises a bacteriostatic composition; said bacteriostatic composition being integrated in said substrate and/or provided as a coating on at least a portion of a surface of said substrate, characterized in that said bacteriostatic composition comprises deferiprone.

2. The medical dressing according to claim 1, wherein said substrate is a foam, an adhesive skin contact layer, or wherein said substrate comprises absorbent gel forming fibers.

3. The medical dressing according to claim 1, wherein said bacteriostatic composition is provided as a coating on a skin-facing surface of said substrate.

4. The medical dressing according to claim 1, wherein said substrate comprises absorbent gel forming fibers, and wherein said absorbent gel forming fibers comprise polyvinyl alcohol (PVA).

5. The medical dressing according to claim 4, wherein said bacteriostatic composition is provided as a coating, and wherein said coating further comprises a non-aqueous solvent selected from the group consisting of methanol, ethanol, n-propanol, iso-propanol, n-butanol, s-butanol, and ethyl acetate.

6. The medical dressing according to claim 1, wherein said bacteriostatic composition is provided as a coating, and wherein said coating comprises one or more cellulosic polymers selected from the group consisting of hydroxypropylmethylcellulose (HPMC), hydroxypropylcellulose (HPC), methylcellulose (MC), and ethylcellulose (EC).

7. The medical dressing according to claim 4, wherein said coating comprises
0.5-14% by weight of deferiprone
1-15% by weight of a cellulosic polymers selected from the group consisting of hydroxypropylmethylcellulose (HPMC), hydroxypropylcellulose (HPC), methylcellulose (MC), and ethylcellulose (EC)
70-98% by weight of a non-aqueous solvent selected from the group consisting of methanol, ethanol, n-propanol, iso-propanol, n-butanol, s-butanol, and ethyl acetate, and
0-15% by weight of water.

8. The medical dressing according to claim 1, wherein said substrate is a foam and wherein said bacteriostatic composition is provided as a coating; said coating comprising a solvent selected from an alcohol, acetate, and an aqueous medium.

9. The medical dressing according to claim 1, wherein said substrate is an adhesive skin contact layer, and wherein said bacteriostatic composition is provided as a coating; said coating comprising an aqueous solvent.

10. The medical dressing according to claim 1, wherein said dressing comprises a backing layer, an adhesive skin contact layer and an absorbent pad arranged between said backing layer and said adhesive skin contact layer, and wherein said substrate is said adhesive skin contact layer; said bacteriostatic composition being provided as a coating on a skin-facing surface of said adhesive skin contact layer.

11. The medical dressing according to claim 10, wherein said bacteriostatic composition of said coating is a first bacteriostatic composition and wherein said absorbent pad and/or said adhesive skin contact layer comprises a second bacteriostatic composition.

12. A method for manufacturing a medical dressing comprising
a) providing a substrate, wherein said substrate is a foam, an adhesive skin contact layer or a substrate comprising absorbent gel-forming fibers,
b) providing a bacteriostatic composition in the form of a solution, suspension or a dispersion, wherein said bacteriostatic composition comprises deferiprone,
c) coating said bacteriostatic composition on at least a portion of a surface of said substrate, and/or
d) integrating said bacteriostatic composition in said substrate.

13. The method according to claim 12, wherein said method further comprises the step of:
drying said coating on the surface of said substrate after said step c).

14. The method according to claim 12, wherein said bacteriostatic composition is coated on at least a portion of a surface of said substrate by means of spray coating.

15. A medical dressing according to claim 1 for use in preventing bacterial infections.

16. Use of deferiprone as a bacteriostatic agent.
